(19) United States
(12) Patent Application Publication
(10) Pub. No.: US 2007/0272239 A1
(43) Pub. Date: Nov. 29, 2007

(54) Dual chamber humidifier

(75) Inventors: Alonzo C. Aylsworth, Wildwood, MO (US); Charles R. Aylsworth, Wildwood, MO (US); Lawrence C. Spector, Austin, TX (US)

Correspondence Address: CONLEY ROSE, P.C.;David A. Rose, P. O. BOX 3267, HOUSTON, TX, 77253-3267, US

(73) Assignee: ACOBA, L.L.C., Chesterfield, MO (US)

(21) Appl. No.: 11/409,646

(22) Filed: Apr. 24, 2006

(51) Int. Cl. A61M 16/00

(52) U.S. Cl. 128/204.17

(57) ABSTRACT

At least some of the illustrative embodiments are a humidifier system comprising an outer housing defining an internal volume, a dividing member within the internal volume that divides the internal volume into a first chamber and a second chamber (the first and second chambers fluidly independent of each other), a first inlet port and first outlet port (the first inlet and outlet ports in fluid communication with the first chamber), and second inlet port and a second outlet port (the second inlet and outlet ports in fluid communication with the second chamber).

### BACKGROUND

Sleep disordered breathing is common throughout the population. Some sleep disorders may be attributable to disorders of the respiratory tract. Sleep apnea may be a disorder where a person temporarily stops breathing during sleep. A hypopnea may be a period of time where a person's breathing becomes abnormally slow or shallow. In some cases, a hypopnea precedes an apnea event.

Although hypopneas and apneas may have multiple causes, one trigger for these type events may be full or partial blockages in the respiratory tract. In particular, in some patients the larynx may collapse due to forces of gravity and/or due to forces associated with lower pressure in the larynx than outside the body. A collapse of the pharynx, larynx, upper airway or other soft tissue in the respiratory tract may thus cause a full or partial blockage, which may lead to a hypopnea or apnea event.

One method to counter collapse of the larynx may be the application of positive airway pressure, possibly by using a continuous positive airway pressure (CPAP) machine. This may be accomplished in the related art by placing a mask over at least the patient's nose, and providing within the mask a pressure communicated to the pharynx, larynx, or upper airway. The pressure within the pharynx, larynx, or upper airway may be greater than the pressure outside the body, thus splinting the airway open. However, forcing air through a patient's nose may cause drying and thus discomfort.

### SUMMARY

Some positive airway pressure devices individually control the airway pressure applied to each naris, which may cause dryness and/or discomfort of differing levels in each naris. To address the dryness and/or discomfort, embodiments of the invention are directed to a dual chamber humidifier, one chamber each for each naris. Having dual chambers enables individually controlling the amount of moisture imparted to the air for each naris. Thus, at least some of the illustrative embodiments are a humidifier system comprising an outer housing defining an internal volume, a dividing member within the internal volume that divides the internal the internal volume into a first chamber and a second chamber (the first and second chambers fluidly independent of each other), a first inlet port and first outlet port (the first inlet and outlet ports in fluid communication with the first chamber), and second inlet port and a second outlet port (the second inlet and outlet ports in fluid communication with the second chamber).

Other illustrative embodiments are a system comprising a positive airway pressure device and a humidifier. The positive airway pressure device comprises a first outlet port that provides positive airway pressure to a first naris of a patient, and a second outlet port that provides positive airway pressure to a second naris of a patient. The positive airway pressure supplied to each naris is individually controlled. The humidifier comprises a first water contact chamber (the first water contact chamber in fluid communication with the first outlet port), and a second water contact chamber (the second water contact chamber fluidly independent of the first contact chamber). The first water contact chamber is configured to fluidly couple to a first naris of a patient, and the second water contact chamber is configured to fluidly couple to a second naris of a patient.

The disclosed devices and methods comprise a combination of features and advantages which enable it to overcome the deficiencies of the prior art devices. The various characteristics described above, as well as other features, will be readily apparent to those skilled in the art upon reading the following detailed description, and by referring to the accompanying drawings.

### BRIEF DESCRIPTION OF THE DRAWINGS

For a detailed description of the preferred embodiments of the invention, reference will now be made to the accompanying drawings in which:

FIG. 1 illustrates a system in accordance with embodiments of the invention;

FIG. 2 illustrates in block diagram form a humidifier in accordance with embodiments of the invention;

FIG. 3 illustrates in block diagram form a heat unit in accordance with the embodiments of the invention;

FIG. 4 illustrates a perspective view of a system in accordance with embodiments of the invention;

FIG. 5 shows a perspective view of humidifier in greater detail and in accordance with embodiments of the invention; and

FIG. 6 illustrates a perspective view of a base unit in accordance with embodiments of the invention.

### NOTATION AND NOMENCLATURE

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”. Also, the term “couple” or “couples” is intended to mean either an indirect or direct connection. Thus, if a first device couples to a second device, that connection may be through a direct connection, or through an indirect connection via other devices and connections.

### DETAILED DESCRIPTION OF THE PREFERRED EMBODIMENTS

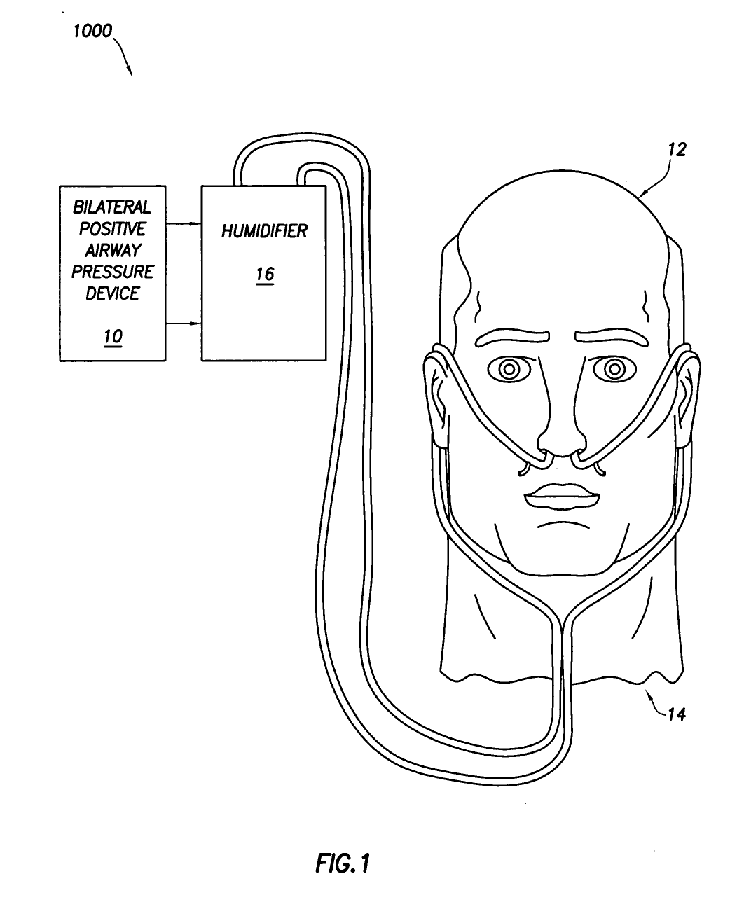

FIG. 1 illustrates a system 1000 in accordance with embodiments of the invention. In particular, FIG. 1 illustrates a bilateral positive airway pressure device 10. A bilateral positive air pressure device such as illustrated in FIG. 1 is a device that applies positive airway pressure to a patient's nares on an individual basis. In this way, each naris is supplied a pressure suited for assisting the patient's breathing through that naris. The bilateral positive airway pressure device 10 may be a device such as described in co-pending patent application Ser. No. 10/851,952, titled “Method and System of Individually Controlling Airway Pressure Application's Nares,” assigned to the same entity as the current specification, and incorporated by reference herein as if reproduced in full below.

The individually controlled pressures are coupled to a patient's nares by way of a nasal mask. For example, FIG. 1 illustrates a patient 12 wearing a mask 14 with two fluidly independent pathways to the patient's nares. However, application of positive airway pressure to a patient's nares may result in dryness and/or discomfort. In order to address these possible difficulties, embodiments of the invention utilize a humidifier 16 coupled between the bilateral positive airway pressure device 10 and the patient 12. As name implies, the humidifier 16 is configured to increase the relative humidity of the air supplied to the patient.

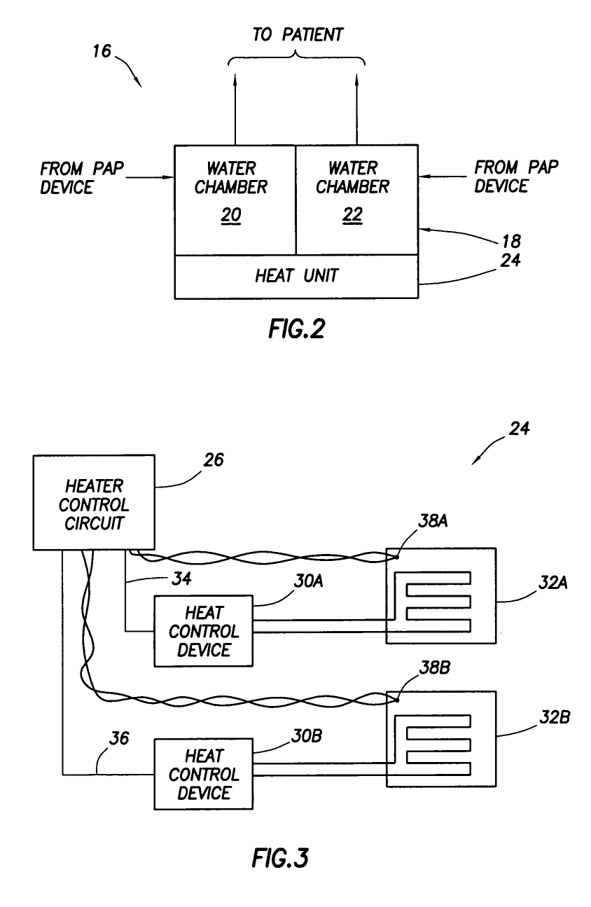

FIG. 2 illustrates in block diagram form a humidifier 16 in accordance with embodiments of the invention. In particular, a humidifier 16 comprises a reservoir 18. Because the humidifier 16 is configured for use with a bilateral positive airway pressure device, the reservoir 18 comprises a first water chamber 20 and second water chamber 22. In some embodiments, allowing air destined for a particular naris to pass over water in its respective water chamber 20, 22 may impart sufficient relative humidity to the air to address dryness and/or discomfort. In alternative embodiments, however, the water within each water chamber 20, 22 may be heated by way of a heat unit 24. In particular, the heat unit 24 may heat the water within each water chamber 22, 24 to approximately 140° degrees Fahrenheit such that the relative humidity of the air leaving the water chamber is higher than the ambient relative humidity.

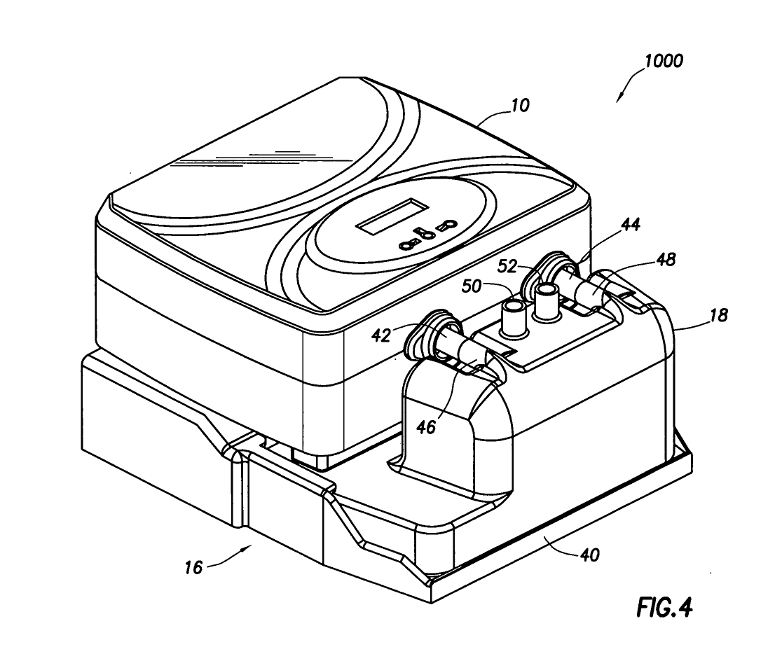

FIG. 3 illustrates in block diagram form a heat unit 24 in accordance with embodiments of the invention. In particular, the heat unit comprises a heat control circuit 26. The heat control circuit 26 couples to heat control device 30A, which in turn couple to heating element 32B. Because in some embodiments humidifier 16 comprises two independent water chambers 20, 22, the heat control circuit 26 also couples to a second heat control device 30B and a second heat element 32B.

The heat control circuit 26 may take many forms. In some embodiments, the heat control circuit 26 is a microcontroller executing software programs to perform the heat control in accordance with embodiments of the invention. In alternative embodiments, the heat control circuit 26 may be an application specific integrated circuit (ASIC), a field programmable gate array (FPGA), or a programmable logic device (PLD). In yet still further embodiments, the control implemented by the heat control circuit 26 may be implemented way of discrete electronic devices. Regardless of the precise nature of the heat control circuit 26, the circuit 26 sends heat control commands to the heat control device 30A across signal line 34. Likewise, the heater control circuit 26 sends heat control commands to the heat control device 30B across signal line 36. The precise nature of the signals propagated across signal lines 34 and 36 is dependant upon the nature of the heat control devices 30. In some embodiments, the 120 volt alternating current (AC) signal available at a wall outlet is applied to the resistive heater elements 32. In order to control the heat generated, however, the heat control devices 30 are selectively activated to only allow portions of the AC signal to be applied to the resistive heater elements 32. In alternative embodiments, direct current (DC) voltages may be applied to the resistive heater elements 32, and in these embodiments the heat control devices 30 control the DC voltage applied to each of the resistive heater elements 32. Other heat control devices may be equivalently used.

Regardless of the precise nature of the heat control devices 30, each resistive heater element 32 also has associated therewith a temperature sensing device 38. The temperature sensing devices 38 may be thermocouples, resistive thermal devices, (e.g. thermistors), or any device capable of measuring temperature in the ranges expected. In some embodiments, each resistive heater element 32 is sandwiched between layers of rubber, with the temperature sensing devices couple to one of the upper or lower rubber members. Other arrangements may be equivalently used.

In accordance with embodiments of the invention, the heater control circuit 26 applies sufficient power to the resistive heater elements 32 (by way of the heat control devices 30) to maintain a predetermined temperature of water in the chambers 20, 22 as sensed by the temperature sensing devices 38. In accordance with at least some embodiments, the heater control circuit 26 attempts to maintain a water or fluid temperature within the reservoir of approximately 140° degrees F. Higher or lower temperatures may be equivalently used. Although a positive airway pressure device 10 may attempt to equalize airflow as between the nares of a patient, in situations where one naris carries significantly less airflow, the individual control of heat within the chamber 20, 22 for that naris keeps the water from overheating, lessens the likelihood of significant condensation forming in the tube supplying air to that naris, and saves energy.

Still referring to FIG. 3, the heater control circuit 26 in accordance with some embodiments also has the ability to sense the operational state of the positive airway pressure device fluidly coupled to the reservoir 18 (FIG. 2) by the amount of power required to maintain the predetermined temperature. In particular, in a situation where there is water in a particular chamber 20, 22 and likewise there is airflow moving through the water chamber 20, 22, a certain amount of power is used to maintain water at the predetermined temperature. In the event the positive airway pressure device ceases airflow to a particular naris (and therefore through a particular water chamber 20, 22), the amount of power used to maintain the water within the water chamber 20, 22 will be less. In accordance with embodiments of the invention, the heater control circuit 26 senses this condition as a no airflow condition, and therefore ceases the water heating in that particular water chamber 20, 22. In a circumstance where airflow to one naris has ceased, airflow to the second naris, however, is most likely operational, and therefore the heater control circuit 26 may continue to sense temperature and heat the water in the remaining water chamber 20, 22.

In yet still further embodiments, the heater control circuit 26 is also capable of sensing when a particular water chamber 20, 22 no longer contains any water. In particular, when all the water from a water chamber 20, 22 has evaporated, the amount of power required to maintain the predetermined temperature (as sensed by the temperature sensor 38) drops significantly from that required to maintain temperature when water is present (whether or not there is airflow through the water chamber 20, 22). In these embodiments, the heater control circuit 26 may cease providing power to the heating element 32 of the water chamber 20, 22 in which all the water has been evaporated. Again, however, the second water chamber 20, 22 may still contain water, thus the heater control circuit 26 may continue to provide power to heat the water in that the remaining water chamber 20, 22.

FIG. 4 illustrates a perspective view of a system 1000 in accordance with the embodiments of the invention. In particular, the system 1000 comprises the bilateral positive airway pressure device 10 fluidly coupled to a humidifier system 16. The humidifier system 16 comprises a reservoir 18 and has a base unit 40. As illustrated, the bilateral positive airway pressure device 10 comprises two outlet ports 42 and 44. It is from these outlet ports 42 and 44 that the bilateral positive airway pressure device 10 provides independently controlled positive airway pressure to couple to the patient's nares. Each of the outlet ports 40, 42 fluidly coupled to inlet ports 46, 48 respectively of the fluid reservoir 18. Airflow from each of the outlet ports 42, 44 of a bilateral positive airway pressure device 10 flows into the respective water chamber and then flows out through the outlet ports 50 and 52 of the water chamber. These outlet ports are configured to fluidly couple to hoses of a nasal mask (such as nasal mask 14 of FIG. 1). The nasal mask in turn couples to each naris of the patient.

The reservoir 18 mechanically couples to the base unit 40. In some embodiments, the reservoir 18 slides into mating relationship with the base unit 40, but other mechanical coupling arrangements may be equivalently used. Thus, in some embodiments the base unit 40 orients the outlet ports 42, 44 of the bilateral positive airway pressure device 10 such that they couple to the inlet ports 46, 48 of the reservoir.

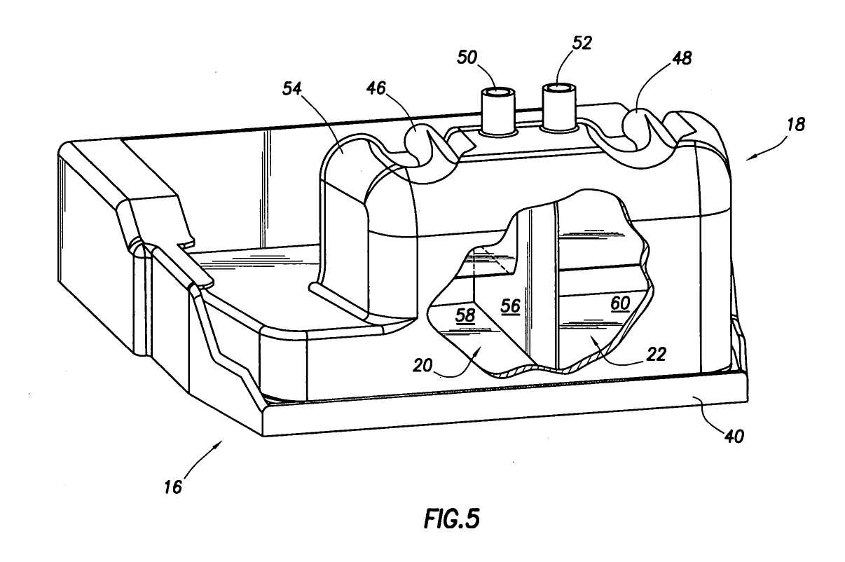

FIG. 5 shows a perspective view of the humidifier 16 in greater detail. In particular, FIG. 5 illustrates the base unit 40 as well as the reservoir 18. However, the reservoir 18 is shown in partial cut-away view to illustrate internal components. The reservoir 18 comprises an outer housing 54 that defines an internal volume. The internal volume defined by the outer housing is divided into first and second water chambers 20 and 22 by way of a dividing member 56. Thus, the reservoir inlet port 46 fluidly couples to the water chamber 20, which in turns fluidly couples to the outlet port 50. Likewise, the inlet port 48 fluidly couples to the water chamber 22, which in turns couples to the outlet port 52.

The water chambers 20 and 22 comprise a bottom portion 58 and 60 respectively. In accordance with at least some embodiments, the bottom portions 58 and 60 are metallic, while the outer housing 54 may be formed of a plastic material. The bottom member 58 and 60 are, in at least some embodiments, separate metallic plates such that the power and heat generated by one resistive heater element is not applied in any significant form to the water and the adjacent water chamber.

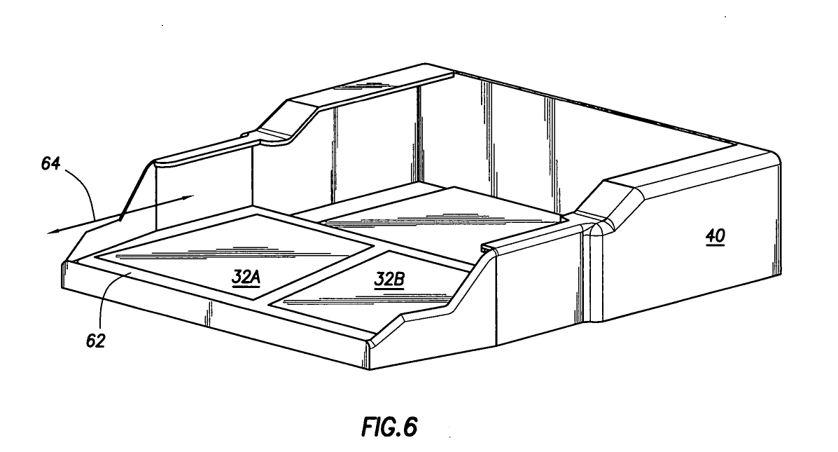

FIG. 6 illustrates a perspective view of the base unit 40 in accordance with the embodiments of the invention. In particular, the base unit 40 comprises bias member 62 that holds the heater elements 32. The bias member 62 biases the heater element 32 upward such that the heater elements are in good contact with the metallic bottom members 58 and 60 (FIG. 5). The bias member 62 may be biased in any suitable fashion, such as by coil springs, leaf springs, or a resilient material. In alternative embodiments, bias member 62 is not used, and instead the heating elements 32 are rigidly affixed to the base unit 40. As implied in FIGS. 4, 5 and 6, the reservoir 18 mechanically couples to the base unit 40 by sliding horizontally into an out of the base unit 40, as illustrated by arrows 64.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A humidifier system for a positive airway pressure device comprising:
an outer housing defining an internal volume;
a dividing member within the internal volume that divides the internal volume into a first chamber and a second chamber, the first and second chambers fluidly independent of each other;
a first inlet port and a first outlet port, the first inlet and outlet ports in fluid communication with the first chamber; and
a second inlet port and a second outlet port, the second inlet and outlet ports in fluid communication with the second chamber.

2. The humidifier system as defined in claim 1 further comprising a base unit, wherein the outer housing is configured to couple to the base unit.

3. The humidifier system as defined in claim 2 wherein the base unit further comprises a heater configured to transfer heat to fluids in first and second chambers.

4. The humidifier system as defined in claim 2 wherein the base unit further comprises:
a first heater, wherein the first heater is configured to transfer heat to fluids within the first chamber; and
a second heater, wherein the first heater is configured to transfer heat to fluids within the second chamber;
wherein the first heater and the second heater are independently controlled.

5. The humidifier system as defined in claim 4 wherein the base unit further comprises:
a first heater control circuit coupled to the first heater; and
a second heater control circuit coupled to the second heater;
wherein each heater control circuit is configured to cease heating when the power draw to heat the fluid in the respective chamber is substantially equal to a no-airflow condition.

6. A system comprising:
a positive airway pressure device comprising:
a first outlet port that provides positive airway pressure to a first naris of a patient; and
a second outlet port that provides positive airway pressure to a second naris of a patient;
wherein the positive airway pressure supplied to each naris is individually controlled;
a humidifier comprising:
a first water contact chamber, the first water contact chamber in fluid communication with the first outlet port; and
a second water contact chamber in fluid communication with the second outlet port, the second water contact chamber fluidly independent of the first water contact chamber;
wherein the first water contact chamber is configured to fluidly couple to a first naris of a patient, and the second water contact chamber is configured to fluidly couple to a second naris of the patient.

7. The system as defined in claim 6 wherein the humidifier further comprises:
a base unit; and
a reservoir portion comprising the first and second water contact chambers;
wherein the reservoir portion couples to the base unit when the contact chambers are in fluid communication with the outlet ports.

8. The system as defined in claim 7 wherein the base unit further comprises a heater that heats fluid within the contact chambers.

9. The system as defined in claim 8 wherein the base unit further comprises:
a first heater, wherein the first heater is configured to transfer heat to fluids within the first chamber; and
a second heater, wherein the first heater is configured to transfer heat to fluids within the second;
wherein the first heater and the second heater are independently controlled.

10. A humidifier system for a positive airway pressure device comprising:
a means for forming an internal volume;
a means for dividing the means for forming into a first chamber and a second chamber, the first and second chambers fluidly independent of each other;
a means for fluidly coupling the first chamber to a means for providing positive airway pressure, and a means for fluidly coupling the first chamber to a breathing airway of a patient;
a means for fluidly coupling the second chamber to a means for providing positive airway pressure, and a means for fluidly coupling the first chamber to a breathing airway of a patient.

11. The humidifier system as defined in claim 10 further comprising a means for accepting the means for forming.

12. The humidifier system as defined in claim 11 wherein the means for accepting further comprises a means for heating a fluid in first and second chambers.

13. The humidifier system as defined in claim 11 wherein the means for accepting further comprises:
a means for heating fluids within the first chamber; and
a means for heating fluids within the second chamber;
wherein each of the means for heating are independently controlled.

14. The humidifier system as defined in claim 13 wherein the means for accepting further comprises:
a means for controlling the means for heating fluid within the first chamber; and
a means for controlling the means for heating fluid within the second chamber;
wherein each means for controlling is configured to cease heating when the power draw to heat the fluid in the respective chamber is substantially equal to a no-airflow condition.
